(19) United States
(12) Patent Application Publication
(10) Pub. No.: US 2011/0081689 A1
(43) Pub. Date: Apr. 7, 2011

(54) Process for Thermal-Mechanical Pretreatment of Biomass

(75) Inventors: Keith C. Flanegan, Rapid City, SD (US); David B. Litzen, Rapid City, SD (US); Dennis A. Harstad, Rapid City, SD (US); James D. Schultze, Rapid City, SD (US)

(73) Assignee: KL ENERGY CORPORATION, Rapid City, SD (US)

(21) Appl. No.: 12/899,441

(22) Filed: Oct. 6, 2010

Related U.S. Application Data
(60) Provisional application No. 61/249,181, filed on Oct. 6, 2009.

(51) Int. Cl. C12P 19/02; C12P 7/10; C12P 1/00

(52) U.S. Cl. 435/105; 435/165; 435/41

(57) ABSTRACT

Disclosed is a process for the thermal-mechanical pretreatment of biomass. The process includes subjecting a biomass feedstock including fibers containing cellulose, hemicellulose and lignin, to thermal reaction under conditions exceeding atmospheric pressure, at a temperature exceeding ambient temperature, at a predetermined moisture content and for a predetermined amount of time. Subsequently, the pressure of said thermal reaction is reduced under conditions resulting in explosive decompression of said biomass. The decompressed biomass is then subjected to axial shear forces to mechanically reduce the size of the fibers of the biomass to obtain treated biomass. The resultant treated biomass has a high level of enzymatic digestability and a low concentration of degradation products.

The present invention is a non-provisional application based on the provisional application Ser. No. 61/249,181 which was filed on Oct. 6, 2009, from which priority is claimed and which provisional application is incorporated by reference herein.

### FIELD OF THE INVENTION

The present invention relates to an environmentally friendly process for the thermal-mechanical pretreatment of biomass involving the pretreatment sequence of thermal reaction followed by mechanical polishing. The thermal reaction involves conveying biomass through a pressurized thermal reactor, followed by steam explosion, and then a multi-zoned compounder which physically breaks down the biomass to effectively and efficiently yield biomass in optimum condition for subsequent enzymatic hydrolysis and conversion.

### BACKGROUND OF THE INVENTION

Largely because of the cost and fluctuations in supply, there is a worldwide interest in finding replacements or substitutes for naturally occurring oil. Much of the interest in replacements for oil is focused upon the production and use of ethanol. The use of ethanol as a part of the supply of motor fuel, for instance, has obvious advantages in reducing dependence upon petroleum-based fuels.

For a variety of reasons, ethanol is currently being produced in quantity largely from grains such as corn or wheat. Such grains naturally contain high concentrations of starches. In the process of converting grains to ethanol, such starches are ordinarily converted to sugars using a number of readily available enzymes. Ethanol is processed from these sugars, chiefly glucose (also known as C6 sugar), using a fermentation process. Although there are other processes, this is currently the most common method of producing ethanol from grains. Under the current state of the art, about 100 gallons of ethanol may be produced from a ton of corn.

Ethanol may also be produced from biomass, which is considered to be any naturally occurring organic material containing cellulose, such as wood waste including slash, pine needles, sawdust, bagasse and any other currently unwanted wood material; but biomass could also include any organic material containing cellulose. Some ethanol is currently being produced from biomass, but such production is significantly more expensive and less efficient than production of ethanol from grains.

Biomass contains cellulose and hemicellulose which may be converted into C6 sugars such as glucose and C5 sugars such as xylose. The structure of these materials in biomass may be considered as a long strand of crystalline cellulose surrounded by a layer of hemicellulose with both the cellulose and hemicellulose surrounded by a layer of what is known as lignin. Hemicelluloses are generally linear or branched polymers of C5 sugars, but may include other compounds. Lignin is a polymeric matrix of aromatic structures.

For biochemical processing, the effective pretreatment of biomass is critical to exposing fermentable sugars to enzymatic hydrolysis. Several forms of biomass pretreatment technologies exist, with many relying on chemical activity to degrade the biomass substrate. Some of these chemical-based pretreatment technologies use mineral acids (mostly sulfuric) or strong alkalines (ammonia) in quantities that require significant neutralization after pretreatment is completed. Other forms of chemical pretreatment include solvent processes that dissolve the lignin fraction of the biomass, leaving the carbohydrates free from lignin interference during enzymatic hydrolysis. Mechanical pretreatment, as in steam explosion, is often used for biomass substrates that have low lignin concentration.

It is typical for pretreatment processes to involve an initial mechanical step in which biomass is comminuted by a combination of chipping, grinding, and/or milling. For instance, steam explosion processes use explosive decompression to significantly reduce the particle size of coarsely chipped biomass, whereas other pretreatment processes commonly employ a secondary grinding or milling step to further reduce the particle size of the chipped biomass. Chipped biomass has a characteristic dimension of 1-3 cm, compared to milled or ground material, which is 0.2-2.0 mm. J. D. McMillan, “Processes for Pretreating Lignocellulosic Biomass: A Review,” National Renewable Energy Laboratory, NREL/TP-421-4978 (November 1992).

Steam explosion (or explosive decompression) is a variation of the high-temperature dilute acid hydrolysis technique in which chipped biomass is treated with saturated steam in a pressure vessel, which is then flashed, causing explosive disruption of the biomass by liberated steam. Typically, steam explosion pretreatments are carried out using saturated steam at 160°-260° C., which corresponds to pressures of 0.69-4.83 MPa (100-700 psia) (Perry et al., Perry's Chemical Engineers' Handbook, 6th edition, 1984) and residence times of tens of seconds to several minutes. Steam explosion has been a highly commercialized pretreatment technique, and numerous reviews of steam explosion are available. Brownell and Saddler, Biotech Bioeng Symp, 14:55-68 (1984), Brownell et al., Biotech Bioeng Symp, 28:792-801 (1986); Clark and Mackie, J Wood Chem Tech, (7:3):373-403 (1987). Generally, both the rate and extent of enzymatic hydrolysis achievable following steam explosion pretreatment increase with increasing duration of treatment. This, however, only refers to washed solids remaining after pretreatment. Because of the formation of degradation products inhibitory to microbial growth, steam-exploded biomass must be washed before enzymatic hydrolysis and subsequent fermentation. The extent and rate of hydrolysis of the solids remaining after pretreatment and washing increase with the severity of the treatment. However, overall saccharification yields fall because washing removes soluble sugars, such as those from hemicellulose hydrolysis. (McMillan, NREL/TP-421-4978, 1992).

Ammonia explosion, or ammonia fiber explosion (AFEX), involves treating a lignocellulosic material with volatile liquid ammonia under pressure, followed by pressure release to evaporate the ammonia and explode the material. In an AFEX pretreatment, ground (1-2 mm) prewetted lignocellulosic material having a moisture content of 0.15-0.30 kg water/kg dry biomass is placed in a pressure vessel with liquid ammonia at a loading of about 1 kg ammonia/kg dry biomass. The ammonia vapor pressure is high (Pvap=1.06 MPa (154 psia) at 300 K), and the pressure becomes quite high in the closed pressure vessel, reaching about 1.24 MPa (180 psia) when the process is carried out at ambient temperature. Dale and Moreira, Biotech Bioeng Symp, (12):31-43 (1982). The mixture is then incubated at the reaction pressure for sufficient time, typically on the order of tens of minutes to an hour, to allow ammonia to penetrate the lignocellulosic matrix. Finally, a valve is opened to flash the reaction mixture to a lower, possibly nonambient, pressure. (McMillan, NREL/TP-421-4978, 1992).

The use of carbon dioxide explosion has also been examined, rather than ammonia- or steam-based explosion, to pretreat alfalfa. Dale and Moreira, Biotech Bioeng Symp, (12):31-43 (1982). Additionally, Puri and Manners, Biotech Bioen (25):3149-3161 (1983), investigated incubating biomass with steam and high-pressure CO₂, and then subjecting the material to explosive decompression. (McMillan, NREL/TP-421-4978, 1992).

Swelling treatments may also be used in biomass pretreatment processes. The degree to which lignocellulosic material swells in the presence of water is increased by treating the material with a swelling agent. Increased swelling is observed to correspond to increased digestibility. Certain swelling agents such as NaOH, amines, and anhydrous NH₃ cause limited swelling, whereas concentrated acids such as H₂SO₄ and HCl or high concentrations of cellulose solvents like cupran, cuen, or cadoxen, totally dissolve (or hydrolyze) holocellulose. Sherrard and Kressman, Ind Eng Chem, 37(1):5-8 (1945); Lin et al., AIChE Symp. Ser. No. 207, 77:102-106 (1981). Although they are powerful solubilization agents for cellulose, however, concentrated acids and metal chelate cellulose solvents have a toxic, corrosive, and hazardous nature, and require high recovery costs. Millett et al., Biotech Bioeng Symp, 6:125-153 (1976). (McMillan, NREL/TP-421-4978, 1992).

Alternative pretreatments include chemical-based pulping processes, supercritical fluid extraction, and supercritical fluid explosion. Regarding chemical-based pulping processes, there are a variety of pulping techniques available, including sulfate, sulfite, and organosolv pulping. Gases such as ozone and oxygen can also be used in pulping operations. However, pulping processes generally suffer from excessive chemical recovery requirements and low yields. In addition, large capital investments are required to install the integrated chemical recovery systems necessary in most chemical-intensive pulping operations. Conventionally, pulping processes are used for producing high-quality paper pulp, or in situations in which other high-value products can be made from byproduct streams, such as furfural (furan-2-carboxaldehyde) and/or other chemicals from xylose or adhesive resins from lignin. (McMillan, NREL/TP-421-4978, 1992).

Supercritical fluid (SCF) treatments can be used to extract chemicals from lignocellulosics. Kiran, J Research at the Univ of Maine, III:2, 24-32 (1987). Many SCF extraction/liquefaction schemes to extract lignins, resins, and waxy materials from lignocellulosics employ solvents that are liquids at room temperature and pressure. For example, McDonald et al., Fluid Phase Equilibria (10):337-344 (1983), treated western red cedar with SCF acetone or methanol at 260°-360° C. and 10-28 MPa (1450-4050 psia), achieving extraction yields of up to 74% by weight. The composition of extracted components was different than expected, however, leading to speculation that degradation was taking place. (McMillan, NREL/TP-421-4978, 1992).

Pretreatment based on incubation in supercritical fluid followed by explosive decompression was evaluated in a study by Castor, SBIR Phase I Final Report on Critical Fluid Comminution of Biomass, DOA Agreement No. 90-33610-5111 (1991). The effectiveness of supercritical carbon dioxide, ethane, ethylene, Freon-22, nitrous oxide, and propane in binary mixture with water was assessed. Hardwood red oak, softwood white pine, and newsprint were used as representative biomass types. Woody materials were tested as chips (10×6×1 mm), cubes (6 mm on each side) and/or shavings (25×6×1 mm), whereas newspaper was tested as strips (6-10 mm wide). Experiments investigated the effects of the following parameters: pressure (up to 69 MPa (10,000 psia)); temperature (up to 200° C.); residence or soak time (1 to 60 min); and biomass moisture content. (McMillan, NREL/TP-421-4978, 1992).

Dilute acid hydrolysis is another aspect of pretreatment processes. When biomass is heated to high temperatures, autohydrolysis of hemicellulose and, to a lesser extent, cellulose occurs, partly catalyzed by acetic acid formed by cleavage of acetyl groups. The catalytic effect of acids on cellulose and hemicellulose hydrolysis is well known, and dilute acid hydrolysis forms the basis of many pretreatment processes. Dilute acid catalysis allows prehydrolysis to be carried out at a lower temperature, thereby reducing sugar decomposition; and dilute sulfuric acid catalysis improves steam explosion saccharification yields. Brownell et al., Biotech Bioeng Symp, 28:792-801 (1986). (McMillan, NREL/TP-421-4978, 1992).

Because of differences in the bonding of compounds in biomass and because of the presence of the lignin sheath, it is much more difficult to process the cellulose and hemicellulose in biomass than it is to process the starches in grains. Most often, an acid hydrolysis process is currently used to extract and reduce the hemicellulose and cellulose to C5 and C6 sugars. Because the process uses sulfuric acid, process equipment such as pumps and pipes must be corrosion resistant and are much more expensive than those used to process grains. The sulfuric acid process also generates a neutralization byproduct, calcium sulfate or gypsum, which must be isolated and disposed of. Prices vary, of course, but a ton of biomass delivered to a processing site costs approximately one half as much as a ton of grain delivered to a processing site. Even though the feed stock costs much less, acid hydrolysis of biomass to ethanol is not generally economically feasible because plant costs are higher than producing ethanol from grains and the yields are lower.

Methods of pretreating biomass to avoid acid processing have also been investigated. For example, U.S. Pat. No. 5,846,787 discloses a process in which cellulose-containing material is pretreated by combining the material with water in a reactor and heating the resultant combination to a temperature of 160° C. to 220° C. while maintaining the pH at 5 to 8. The resultant material may then be hydrolyzed using enzymes Consequently, the process in U.S. Pat. No. 5,846,787 may be effective when applied to herbaceous feedstocks, but not to woody biomass. Furthermore, a lack of mechanical polishing in this prior process would result in process slurries that are only manageable if significantly diluted. Also, prior acid and steam explosion processes may be limited to 8% to 15% by weight solids based on the total weight of the slurry, whereas the process of the present invention can achieve over 25%, saving significant operating and capital costs.

The present invention is believed to solve, in a unique and effective manner, various problems related to the use of biomass for production of ethanol, including the problems and disadvantages described above.

### SUMMARY OF THE INVENTION

The present invention relates to a method of converting biomass to monomeric sugars involving thermal pretreatment of the biomass followed by extrusion in a reactor that physically breaks the biomass down such that it may be enzymatically processed to produce monomeric sugars. Specifically, the present invention provides a process for thermal-mechanical pretreatment of biomass that results in biomass having a high level of enzymatic digestability and a low concentration of degradation products compared to biomass pretreated according to prior art methods.

Importantly, the process of the present invention can convert biomass (especially wood and wood waste materials) to ethanol or other products derived from monomeric sugar fermentation using little or no acids. This process therefore has a significantly lower impact on the environment as compared to other technologies being used for the production of ethanol from biomass, making the present invention highly advantageous.

Another significant advantage of the present invention is that the biomass may be pretreated such that the cellulose and hemicellulose contained is easily and efficiently converted to ethanol using enzymatic hydrolysis without any significant formation of biomass conversion inhibitors (such as furfural) that are potentially toxic to yeast or other fermentation organisms. Additionally, the pretreatment process of the present invention advantageously eliminates the need for corrosion resistant equipment necessary for acid hydrolysis and similar processes. The pretreatment process of the present invention also advantageously uses materials that are inexpensive, easily handled, and environmentally safe in order to exclude the need for neutralizing the process and disposing of neutralization byproducts.

According to one embodiment, the present invention is directed to a process for the thermal-mechanical pretreatment of biomass. The process includes subjecting a biomass feedstock including fibers containing cellulose, hemicellulose and lignin, to thermal reaction under conditions exceeding atmospheric pressure, at a temperature exceeding ambient temperature, at a predetermined moisture content and for a predetermined amount of time. Subsequently, the pressure of said thermal reaction is reduced under conditions resulting in explosive decompression of said biomass. The decompressed biomass is then subjected to axial shear forces to mechanically redue the size of the fibers of the biomass to obtain treated biomass. The resultant treated biomass has a high level of enzymatic digestability and a low concentration of degradation products.

### BRIEF DESCRIPTION OF THE DRAWINGS

FIG. 1 is a process flow diagram showing a thermal-mechanical pretreatment process according to the present invention.

FIG. 2 is a schematic of a compounder (twin screw) that may be used in the process of the present invention.

FIG. 3 is a plot of total evaporator area vs. slurry loading according to an embodiment of the present invention. The data show that as slurry concentration is increased, less evaporator capacity is necessary. In particular, this is shown in terms of heat exchanger area needed to process the same amount of biomass, but over a range of increasing slurry concentrations.

FIG. 4 is a plot of steam rate vs. slurry loading, according to an embodiment of the present invention. The data show that as slurry concentration is increased, less steam energy is needed to drive the conversion to cellulosic ethanol.

FIG. 5 is a plot of furfural to biomass ratio vs. vent steam to dry biomass ratio, according to an embodiment of the present invention.

FIG. 6 is a plot of acetic acid to biomass ratio vs. vent steam to dry biomass ratio, according to an embodiment of the present invention.

FIG. 7 is a schematic of a compounder design according to one embodiment of the present invention.

FIG. 8 is a plot of glucan yield vs hydrolysis time under various treatment conditions.

FIG. 9 is a plot of xylan yield vs hydrolysis time under various treatment conditions.

FIG. 10 is a plot of percentage of digestion of various hydrolyzed sugars vs. increased combined severity factor (CSF).

FIG. 11 is a plot of percentage of acetate formation vs. increased combined severity factor (CSF).

FIGS. 12(a), (b) and (c) are photomicrographs of fibers treated according to an embodiment of the present invention. Photos are taken via a Scanning Electron Microscope (SEM), aligned in progressively less magnification. Multiple layers of cell wall are effectively exposed by the application of axial shear. FIGS. 12(a) and (b) show cellulosic fibers that have been ‘stripped’, revealing the inner portions of the cell wall where the majority of the hydrolyzable carbohydrates reside. With less magnification, FIG. 12(c) shows the large amount of surface area exposed as a result of axial shear application.

FIGS. 13(a), (b) and (c) are photomicrographs of control fibers treated by hammer-milling to a median particle size of 150 microns. Photos are taken via a Scanning Electron Microscope (SEM), aligned in progressively less magnification. As a contrast to FIG. 12, cell wall surfaces are virtually unaffected by hammermilling, observing the survival of the ‘pit membranes’ (holes) and the smooth surfaces. The smooth outer cell wall layer (lignin), protects the remaining substrate from enzymatic attack

### DETAILED DESCRIPTION OF THE PREFERRED EMBODIMENTS

In the process of the present invention, biomass is thermally pretreated and then subjected to a mechanical treatment in a compounder before being enzymatically processed to produce ethanol. As used herein, “biomass” refers to any organic material that contains cellulose and/or hemicellulose—e.g including but not limited to herbaceous and agricultural products (such as species of alfalfa, bamboo, citrus peels, corn cob, corn stover, miscanthus, rice straw, sugarcane bagasse, sugar beet pulp, switchgrass, wheat straw, and the like), and hardwoods (such as species of ash, aspen, basswood, beech, birch, cottonwood, elm, eucalyptus, hickory, mahogany, maple, oak, poplar, walnut, willow, and the like), and softwoods (such as species of cedar, fir, hemlock, juniper, pine, spruce, and the like) and combinations thereof.

In one embodiment of the present invention, wood chips (e.g., approximately 1″×1″×¼″ in dimension having their natural moisture content (typically 25% to 50% by weight) are conveyed to a chip bin (e.g., by a conveying means such as a transfer screw conveyor) in such a manner as to generate an inventory sufficient to continually supply a plug feeder (or rotary valve). The function of the plug feeder (or rotary valve) is to convey wood into a pressurized thermal reactor. Preferably, the thermal reactor operates at about 150° C. to about 200° C. (about 70 psia to about 225 psia, respectively provided by a live steam injection. More preferably, the thermal reactor operates at about 175° C. to about 195° C. (about 130 psia to 200 psia, respectively). However, the operating temperature of the thermal reactor is highly dependent on the type of feedstock. For instance, herbaceous feedstocks require lower temperatures than woody biomass.

In one embodiment, the steam injection is provided at a minimum pressure of 290 psig and reduced adiabatically to the thermal reactor operating pressure, therefore allowing the steam to enter the reactor slightly superheated in order to compensate for any ambient heat loss in the reactor. Generally, the higher the steam pressure, the more superheat can be transferred to the reactor.

The function of the thermal reactor is to thermally degrade a major portion of the hemicellulose fraction of the biomass by providing sufficient residence time (e.g., about 10 minutes to about 90 minutes, and preferably about 30 minutes to about 60 minutes) at the stated conditions without adding significant condensed moisture to the biomass. The moisture content of the biomass undergoing thermal reactor treatment should be adjusted to a range from about 40 to about 80%, preferably from 50 to 75% and ideally from 60 to 75%.

For more difficult substrates (like softwoods), the reactor pH can be lowered to a suitable range (e.g., about 1.0 to about 6.0, and preferably about 2.5 to about 4) by injecting a small amount of a mineral acid (e.g., sulfuric, nitric, phosphoric or hydrochloric) or an organic acid (e.g., acetic, or lactic), thereby improving hemicellulose conversion kinetics. Degradation of hemicellulose is necessary to sufficiently perforate the substrate to increase the enzymatic hydrolysis yield of the cellulose, and to substantially soften the substrate.

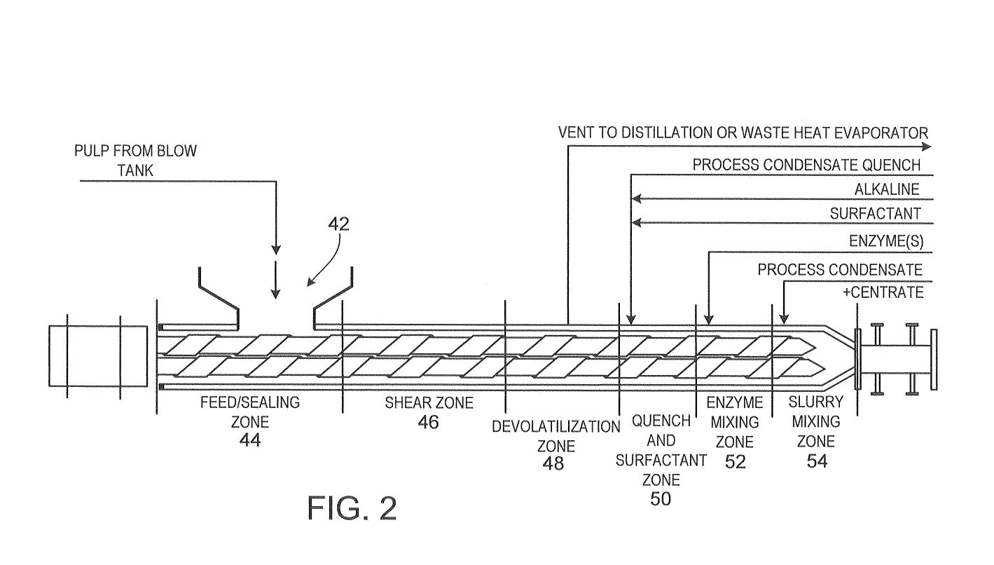

From the thermal reactor, pressure is dramatically reduced by explosive decompression in a single step (e.g., to a pressure of about 5 psia to about 32 psia, preferably about 15 psia to about 32 psia, more preferably to about 30 psia to about 32 psia) which cools the reacted biomass (e.g., to a temperature of about 70° C. to about 125° C., and preferably about 120° C. to about 125° C.) by recovering the steam flash in a flash tank 6. This recovered steam is stripping steam that is directed either to a downstream distillation column or a waste heat evaporator. The cooled biomass is then conveyed to a compounder, such as a twin screw co-rotating compounder, a twin screw counter-rotating compounder, or a single screw compounder, the twin screw co-rotating compounder being preferred. Referring to FIG. 2 as a generic application of the twin screw co-rotating compounder, the compounder has several zones, the first of which is a feed zone. The next zone is a shear zone, which allows the compounder to initially function as a mechanical polisher by imposing shear along the longitudinal axis of the biomass fibers in specially designed compounder screw elements. It has been determined by the present inventors that axial shear (shear applied to the length of the biomass fiber) imposed by the compounder on thermally untreated raw wood provides significantly improved pretreatment when compared to results obtained for hammer-milled wood flour.

According to the present invention, it has been shown that the pretreatment sequence of thermal reaction followed by mechanical polishing (or fiber size reduction) is the most effective combination to maximize enzymatic hydrolysis conversion. When compared to thermally untreated biomass, the inventors found between 75% and 95% mechanical power reduction when the biomass is first thermally pretreated.

Following the treatment of the biomass in the shear zone of the compounder, it is desirable to reduce the temperature of the biomass to temperatures suitable for enzymatic treatment and subsequent fermentation. Thus, subsequent to the shear zone, devolatilization is provided to remove the heat of frictional energy generated in the shear zone. Steam generated in the devolatilization zone is removed from the compounder, combined with steam generated from the flash tank, and recovered as stripping steam in a distillation column or waste heat evaporator. Following the devolatilization zone, the biomass is further quenched with recycled process water to reduce the biomass temperature (e.g., to about 95° C.). At this point, additives such as nitrogen-based alkalines (e.g., aqueous ammonia and the like) and surfactants such as one or more non-ionic surfactants (e.g., corn steep liquor or a polysorbate, such as Tween 80) are added in a precision mixing zone of the compounder (i.e., a quench zone or a quench and surfactant mixing zone). Any alkaline additive can be used, but nitrogen-based alkalines are preferred because they provide a double benefit in that the additive will also provide necessary nitrogen to keep fermentation yeast healthy.

The alkaline additive is intended to bring the biomass pH to an optimal level for later enzymatic hydrolysis. Preferably, the pH is adjusted to about 4.5 to 5.5, most preferably 5.0, at this point. The surfactant additive is intended to improve enzyme efficiency, which relates to the amount of enzyme required to achieve a predetermined level of hydrolysis conversion. The technology of the present invention demonstrates high enzyme efficiency because a very high percentage of cellulose conversion to glucose can be achieved with very low enzyme usage. That is, at least 80% (preferably at least 90% and most preferably 100%) glucose recovery can be achieved. In one embodiment, about 80-90% glucose recovery may be achieved. In another embodiment, 100% glucose recovery may be achieved. High glucose recovery is one of several aspects of the present invention that provide for significant economic savings.

Downstream of the quench zone, enzymes are mixed with the biomass in a precision mixing zone (i.e., an enzyme mixing zone) where residence time is minimized to prevent thermal denaturing of the enzymes. Finally, the biomass is fully slurried in a slurry mixing zone by adding recycled process water to the compounder, which thus produces a slurry stream that has been pH and temperature adjusted for optimum enzymatic hydrolysis.

In addition to improving hydrolysis conversion in biomass, mechanical polishing, or grinding, also effectively reduces the biomass fibers to a size that can be slurried with water at higher consistencies than is possible using other pretreatment technologies alone, such as dilute acid or steam explosion. Whereas slurry solids are limited to 15% by weight solids based on the total weight of the slurry, slurries having more than 25% by weight solids based on the total weight of the slurry can be achieved with the combination of thermal and mechanical pretreatment according to the present invention. Both capital and operating cost savings are significant when 25% by weight solids slurry processing is compared to 15% by weight solids slurry processing.

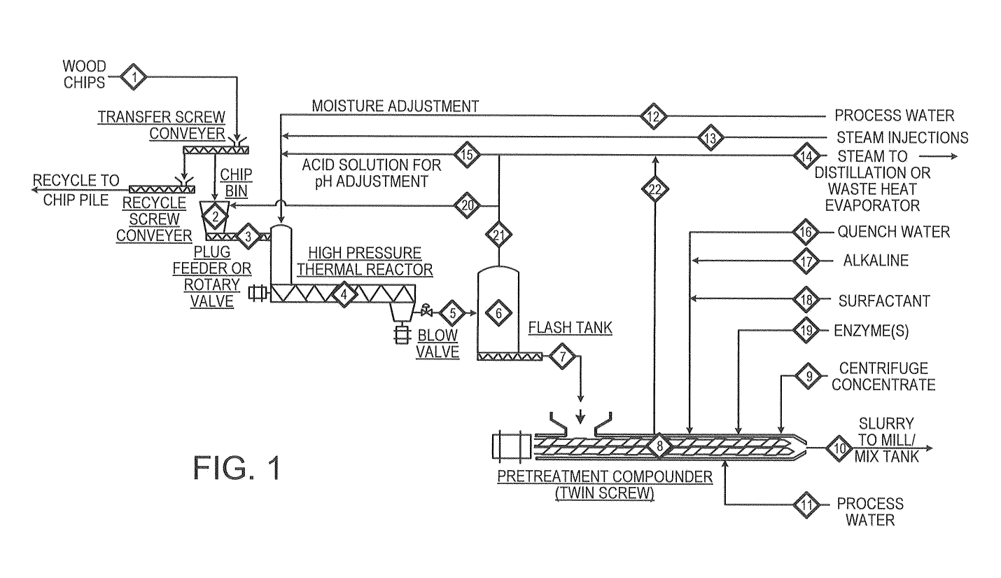

Referring to the figures, FIG. 1 illustrates an embodiment of the process of the present invention, wherein wood chips in stream 1 are conveyed to chip bin 2 via a transfer screw conveyor, which feeds into plug feeder (or rotary valve) 3. By this part of the process, debris removal is essentially complete. The recycle screw conveyor helps ensure the chip bin remains sufficiently full (and thereby avoids losing the plug in the plug feeder by losing feed). In other words, the transfer screw conveyor feeds more biomass than plug feeder (or rotary valve) 3 is feeding, recycling the excess. Plug feeder (or rotary valve) 3 moves wood into thermal reactor 4, which operates at a temperature and pressure provided by live steam injection 13. The pH of the biomass inside thermal reactor 4 can be adjusted by injecting acidic solution through stream 15. Process water can be added to thermal reactor 4 through stream 12. Pressure in thermal reactor 4 is reduced in a single step using a blow valve and stream 5. Thermally reacted biomass is cooled in flash tank 6 by recovering the steam flash through stream 21. Cooled biomass is then conveyed through stream 7 to pretreatment compounder 8 (e.g., a twin screw pretreatment compounder). The biomass passes through a feed zone of compounder 8, followed by a shear zone, after which steam is generated in a devolatilization zone and released through stream 22. The steam released into stream 21 (from the flash tank) and stream 22 (from the compounder) combines in stream 14 and is recovered as stripping steam directed to a distillation column or waste heat evaporator (not shown). After the devolatilization zone, biomass is quenched in the quench zone in compounder 8 using recycled process water provided through stream 16. Additives can then be added into compounder 8, such as aqueous ammonia through stream 17 and surfactant through stream 18. Downstream of the quench zone, enzymes are added through stream 19 into the enzyme mixing zone in compounder 8. Finally, the biomass in compounder 8 is fully slurried by adding recycled process water through streams 9 and 11, resulting in a slurry that flows through stream 10, which has been pH and temperature adjusted for optimum enzymatic hydrolysis.

FIG. 2 illustrates a schematic embodiment of a compounder 40 that may be used in the process of the present invention. In this embodiment, the compounder comprises a plurality of zones, which can be in a particular order. As shown in FIG. 2, the first zone in the compounder is a feed/sealing zone 44 where pulp from a blow tank first enters the compounder at hopper 42, and is conveyed by the compounder through successive treatment zones from left to right as shown in the schematic. Next is a shear zone 46 where pulp is mechanically polished by shear imposed along the longitudinal axis of biomass fibers, creating frictional heat. Next is a devolatilization zone 48 where the frictional heat from the shear zone is released in the form of vented steam, reducing the temperature of the biomass. Next is a quench and surfactant mixing zone 50 where the biomass is further cooled by, for example, addition of recycled process water (process condensate quench). Adjustment of pH and/or addition of surfactant(s) may also occur in the quench and surfactant mixing zone. Next is an enzyme mixing zone 52 where enzymes are added during a minimized residence time. The enzyme mixing zone is followed by a slurry mixing zone 54 where the biomass is cooled even further by, for example, addition of recycled process water. The resulting slurry stream is then in optimal condition for subsequent enzymatic hydrolysis and production of ethanol.

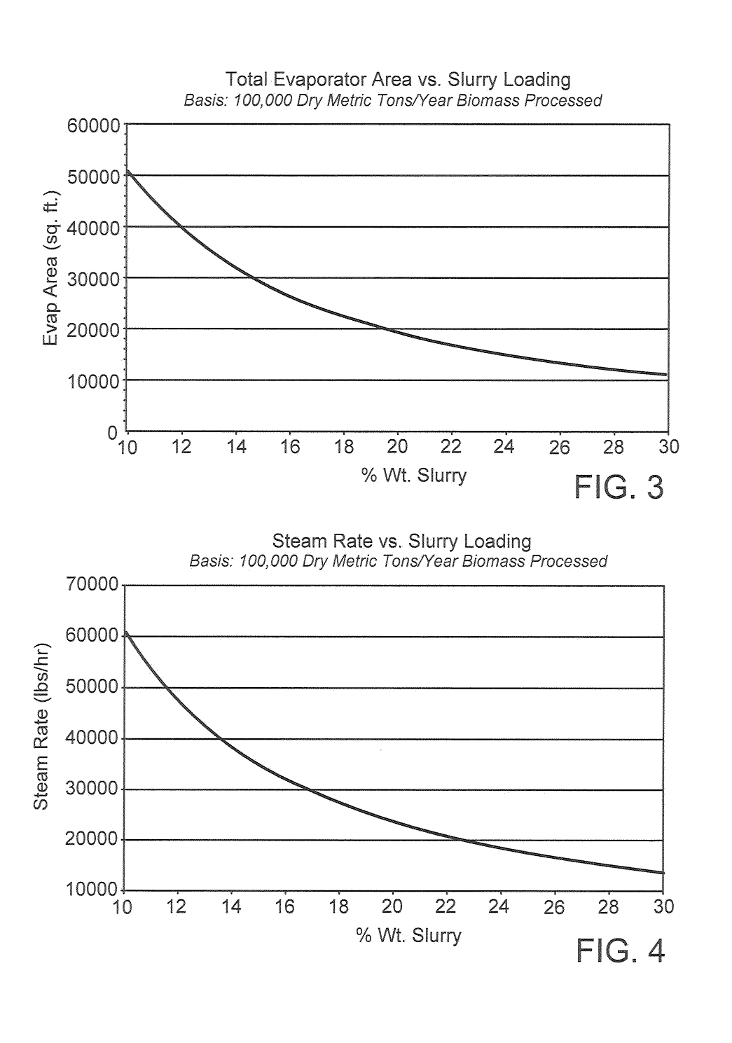

FIG. 3 illustrates the capital savings realized by the process of the present invention for the evaporator, a major cost item in the ethanol production process. The difference in total evaporator heat transfer area required to recover water from a process that maintains a 25% by weight solids fermentation slurry is roughly 50% less than the area required for a 15% by weight solids fermentation slurry, and roughly 70% less than the area required for a 10% by weight solids slurry. This relationship affects all equipment between and including the hydrolysis tank and the distillation column, resulting in significant capital cost savings.

FIG. 4 shows the effect of slurry concentration on steam savings, another significant operating cost for an ethanol production plant. Almost identical, the difference in plant steam usage required to operate a process that maintains a 25% by weight solids fermentation slurry is roughly 50% less than the steam required for a 15% by weight solids fermentation slurry, and roughly 70% less than the area required for a 10% by weight solids slurry. The total plant steam savings results in a significant operating cost reduction for the ethanol plant.

These benefits illustrated in FIGS. 3 and 4 are provided by the process of the present invention in addition to the effective, environmentally friendly means of increasing the effectiveness of the hydrolyzing enzymes set forth herein.

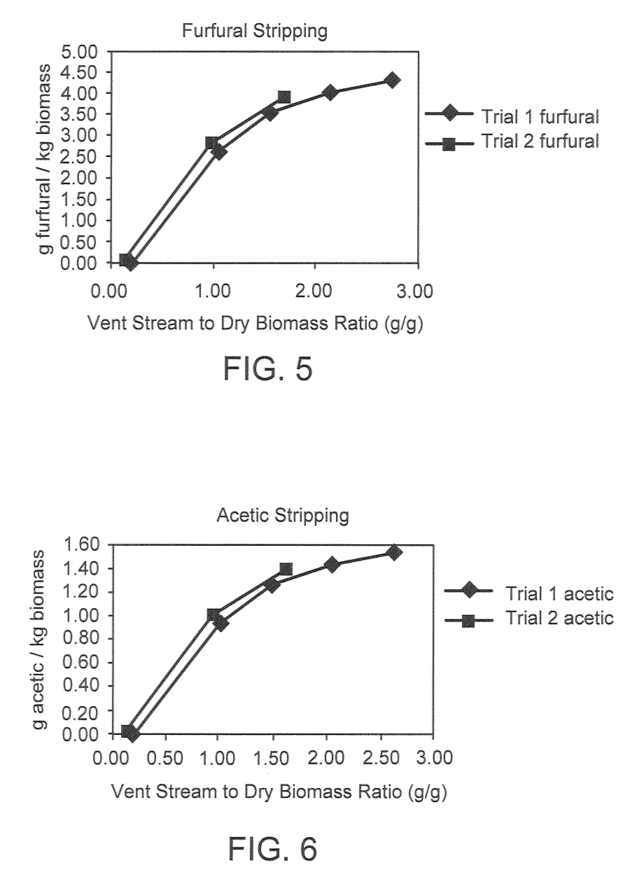

As a result of necessarily removing the steam energy from the thermal reaction and mechanical steps, the described process (FIG. 1, stream 14) also performs an important function of removing reaction degradation products, such as furfural, (which forms a low-boiling azeotrope with water), acetic acid, and other hydrophobic biomass extracts which are harmful or inhibitory to fermentation organisms. Referring to FIGS. 5 and 6, 85% or more of the furfural, and 30% or more of the acetic acid is removed from the biomass prior to slurry hydrolysis and fermentation. Furthermore, the heat from the thermal step can be easily recovered into the downstream ethanol recovery process while isolating the recovered inhibitory compounds, potentially purified into value-added byproducts of the ethanol production process.

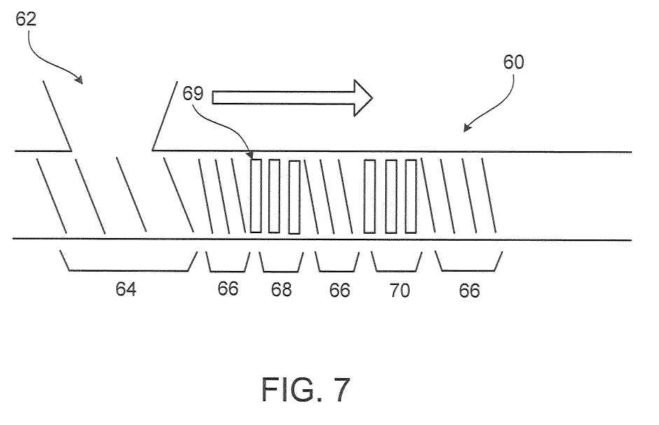

FIG. 7 shows a schematic of one compounder element design. The primary function of the compounder is to apply a shear force along the length of the biomass fiber, effectively stripping the outer layer of the cell wall consisting mostly of lignin, exposing carbohydrates such as glucan (cellulose), xylan, mannan to enzymatic attack.

Referring to FIG. 7, from left to right, biomass is fed into the compounder 60 at feed throat 62 where conveying elements 64 move the biomass with elements having progressively increasing pitch 66 to compress and push the biomass into the first high shear pretreatment zone 68. The high shear pretreatment zone consists of a series of kneading and high surface area elements 69 that specifically apply force to the longitudinal axis of the biomass fiber. A second set of progressive conveying elements 66 follows the first high shear zone, preventing the biomass from overheating or burning due to frictional forces. The second set of conveying elements 66 lead to a secondary high shear zone 70 that further strips the biomass fibers with the use of kneading and high surface area elements. Finally a third set of conveying elements 66 is used to move the mechanically pretreated biomass out of the compounder. The two high shear zones not only provide effective mechanical pretreatment without damaging (burning) the organic fiber, but also provide mechanical balance to the compounding unit. For all examples below, the compounder was used only to provide mechanical shear and did not function to devolatilize, mix, or quench as generally described above.

### Biomass Feedback Testing Protocol

The following feedstock test protocol was used in all examples below (unless otherwise noted):
Step 1: Determination of Feedstock Composition—Feedstock was sent to a third party accredited laboratory to analyze the biomass composition via industry-accepted National Renewable Energy Laboratory (NREL) methods and procedures.
Step 2: Thermal Reaction Optimization—The biomass was knife-milled or hammer milled to a particle size that allowed insertion of the biomass into a 0.75 inch diameter, 12 inch long reactor tube (typically milled to 0.125 inch minus). The biomass moisture was adjusted to 40-80%, depending on experiment objectives. Up to 40 reactor tubes were filled with milled, moist biomass and exposed to a range of temperature, residence time, and pH conditions and subsequently hydrolyzed enzymatically to statistically determine the optimum total fermentable sugar yield.
Step 3: Pretreatment Scale-Up At Optimum Thermal Conditions—Biomass was pretreated by duplicating the optimum condition (or range of probable optimum conditions) of the previous set of experiments described in Step 2, in a 24-liter, live-steam batch reactor. The reactor product was subsequently pretreated mechanically in a 25 millimeter co-rotating twin screw compounder.
Step 4: Enzyme Response and Preliminary Fermentation—Pretreated material from Step 3 was enzymatically hydrolyzed in 250 milliliter shake flasks with aqueous slurries ranging from 2% w/w to 20% w/w using a range of enzyme concentrations, resulting in an enzyme response curve. Initial small scale (250 milliliter shake flasks) fermentations were also conducted to screen for fermentation organism inhibition.
Step 5: Final Hydrolysis and Fermentation—larger scale hydrolyses and fermentations (7 liter agitated vessels) were performed to determine final ethanol yield potential at target enzyme and slurry loadings.

### EXAMPLES

The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the appended claims, along with the full scope of equivalents to which the claims are entitled.

### Example 1

### Control Testing

Control Sample 1: Untreated sawdust was determined to have an average glucose recovery after enzymatic hydrolysis of 3.09% with a margin of error of plus or minus 0.38%. That is, 3.09% of the available glucose was recoverable from the untreated sawdust. No pretreatment was used for this control sample, other than coarse size reduction.

Control Sample 2: Untreated wood flour (pulverized sawdust) was determined to have an average recovery of 7.69% with a margin for error of plus or minus 0.15%. No pretreatment was used for this control sample, other than hammer milling used to grind the feedstock into a very fine wood flour.

The average glucose recovery in these control samples is very low, even when milling the material to a fine flour (median particle size=150 microns), which requires very high energy per unit mass biomass processed. In contrast, the process of the present invention can, with significantly less power than conventional milling, achieve significantly higher glucose recovery percentages, as illustrated in Example 2.

The following experiments illustrate embodiments of the thermal-mechanical pretreatment process of the present invention.

### Example 2

### Thermal-Mechanical Pretreatment Testing

Lodgepole Pine: In this experiment, Lodgepole pine was processed through a hammer mill to reduce the particle size to less than 0.125 inches, after which the moisture content of the particles is adjusted to 60%. Using small reaction tubes and an experimental design (Step 2 in the testing protocol) to vary reaction severity (a combination of conditions, or combined severity factor, that includes thermal reactor residence time, temperature, and pH), a determination was made via statistical analysis as to which reaction severity result maximizes overall fermentable sugar yield. Combined severity factor, or CSF, is defined as: CSF=log 10 (residence time*exp[(temperature-100)/14.75])−pH, where residence time is in minutes and temperature is in degrees Celsius. Next, the biomass was directed into a larger scale (22.5 liter) thermal pretreatment reactor at conditions defined as optimum by the experimental design described above (approximately 140 psig with a residence time of about 1 hour). Homogenous material exiting the thermal pretreatment reactor was collected, transferred and fed into a 25 mm twin screw co-rotating mechanical compounder. The compounder was operated at 500 RPM and 25° C. The material exiting the compounder was collected, enzymatically hydrolyzed and analyzed Sugar and ethanol concentrations were measured directly via high performance liquid chromatography (HPLC). The results are shown in Table 1 below:

TABLE 1
Lodgepole Pine

| Parameter | Measurement |
|---|---|
| Average enzyme dosage FPU/g cellulose | 30.02 |
| Average enzyme dose % (wt/wt) | 19.23% |
| Average hydrolysis moisture (%) | 71.23% |
| Average cellobiose recovery (%) | 0.00% |
| Average glucose recovery (%) | 53.61% |
| Average xylose recovery (%) | 40.50% |
| Average galactose recovery (%) | 0.00% |
| Average arabinose recovery (%) | N/A |
| Average mannose recovery (%) | N/A |
| Average HMF recovery (%) | 0.00% |
| Average furfural recovery (%) | 0.00% |

Side-by-side testing and sampling was conducted as well for processes involving: (i) hammer milling alone; (ii) hammer milling and mechanical compounding; (iii) hammer milling and thermal reacting; and (iv) hammer milling, thermal reacting, and mechanical compounding. The results showed that glucose recoveries at 30 FPU/g for hammer milling alone were poor (3%). (“FPU” is defined as a Filter Paper Unit, a term used by enzyme producers to define enzyme strength.) The results for hammer milling plus thermal reacting (22%) and hammer milling plus mechanical compounding were both somewhat better (39%), and for hammer milling, thermal reacting, and mechanical compounding (according to the present invention) were very good (54%).

Still, in this test, the thermal reaction step was operated at non-optimal conditions due to a steam pressure limitation that was used only to illustrate the relative impact of each pretreatment step in the protocol. Also, an non-optimized compounder screw element design was used in the mechanical pretreatment step. Using optimized thermal conditions and mechanical screw element designs, glucose recovery can reach 80-90% (or even higher), as illustrated below in the processing of bagasse, eucalyptus, mixed hardwood, and mixed softwood feedstocks.

### Example 3

Bagasse: A biomass feedstock of sugar cane bagasse was processed using the foregoing protocol, except that the feedstock was not subjected to initial milling The material exiting the compounder was collected and analyzed, and the results are shown in Table 2 below (ODM is defined as Oven Dry Matter):

TABLE 2
Bagasse

| | |
|---|---|
| Design Parameter | Bagasse |

Thermal pretreatment design targets

| | | |
|---|---|---|
| Temperature | ° C. | 180 |
| Residence time | min | 17.5 |
| Additives | | H₂SO₄ |
| Additive application rate | mg/g ODM | 2 |
| Reactor discharge | wt/wt | 70% |
| moisture | | |

Mechanical pretreatment design targets

| | | |
|---|---|---|
| Compounder speed | rpm | 700 |

Enzymatic hydrolysis targets

| | | |
|---|---|---|
| Slurry concentration | g ODM/g slurry | 17% |
| Hydrolysis time | Hours | 72 |
| Temperature | ° C. | 50 |
| pH | | 5.3 |
| Surfactant type | | Not applicable |
| Surfactant dosage | mg/g ODM | 0 |
| Enzyme type | | celllulases |
| Enzyme protein dosage | mg/g ODM | 4 |
| Hydrolysis yields | | |
| achieved¹⁾ | | |
| Glucan yield achieved | % of theoretical | 79% |
| Xylan yield achieved | % of theoretical | 81% |

Fermentation targets

| | | |
|---|---|---|
| Slurry concentration | g ODM/g slurry | 17% |
| Fermentation time | hours | 48 |
| Fermentation temp | ° C. | 32 |
| Fermentation pH | | 5.3 |
| Fermentation nutrients | | |
| Peptone dosage | g/l broth | 5.2 |
| Yeast extract dosage | g/l broth | 2.6 |
| Yeast dosage | g/l broth | 1.0 |
| Final ethanol titer | g/l | 42.4 |
| Fermentation yield | % theoretical, | 90.3% |
| | based on initial | |
| | sugars | |
| Ethanol yield | l/BDMT | 317.5 |

Sugar and ethanol concentrations were measured directly via high performance liquid chromatography (HPLC). Sugar yield calculations were made using the NIST average bagasse composition. Fermentation yield was calculated using hydrolyzate sugar concentrations. Ethanol yield was calculated using the ethanol concentration and slurry mass percentage.

Side-by-side testing and sampling was conducted as well for processes involving: (i) mechanical compounding; (ii) thermal reaction only and (iii) thermal reaction plus mechanical compounding. In these trials, the hydrolysis slurry was 2 wt % total solids and no initial milling was done before either thermal reaction or compounding. The results are found in the Table 3 below:

TABLE 3
Bagasse (No Milling)

| | Glucose | Xylose |
|---|---|---|
| Pretreatment Process | Recovery (%) | Recovery (%) |
| Mechanical Compounding Only | 2% | 17% |
| Thermal Reaction Only | 56% | 53% |
| Milling + Thermal Reacting + | 76% | 61% |
| Mechanical Compounding | | |

In this particular set of tests, the power reduction measured between milling/mechanical compounding and milling/thermal reacting/mechanical compounding was 95.1% (21.0 kilowatt-hours per kilogram versus 1.03 kilowatt-hours per kilogram).

### Example 4

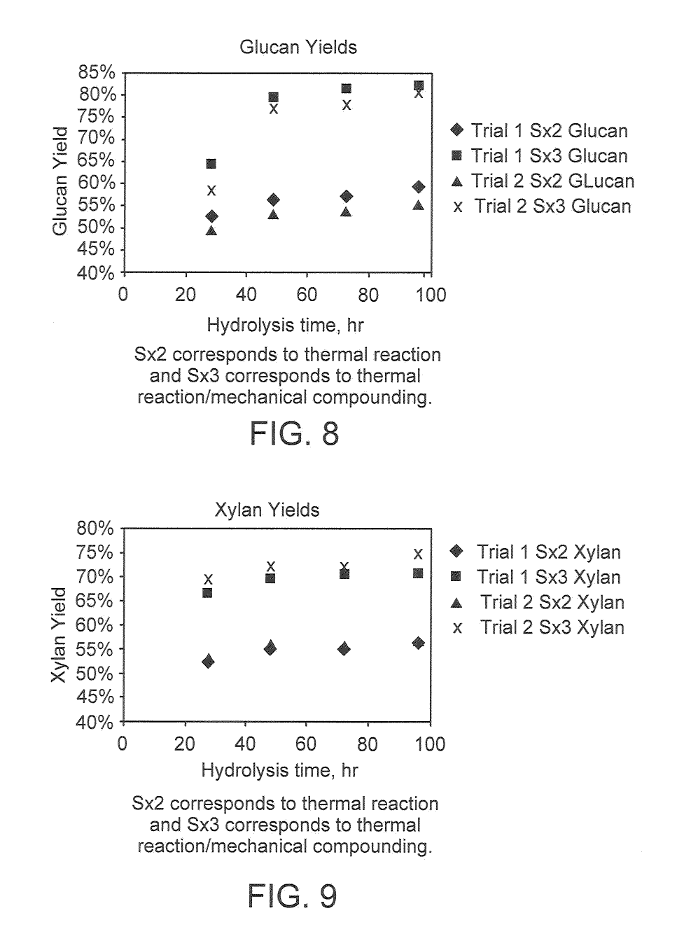

Additional experimental data specifically isolating the processes of milling/thermal reacting versus milling/thermal reacting/mechanical compounding indicates a significant improvement of the present invention over the milling/thermal reaction process. In FIGS. 8 and 9, bagasse was pretreated via the isolated and combined pretreatment processes described above and analyzed over a period of hydrolysis time up to 100 hours, showing clearly the synergy of the thermal-mechanical pretreatment combination compared to each pretreatment method performed individually.

### Example 5

Eucalyptus: Biomass feedstock of eucalyptus (a hardwood) was processed using the foregoing protocol. The material exiting the compounder was collected and analyzed, and the results are shown in Table 4 below:

TABLE 4
Eucalyptus

| | |
|---|---|
| Design Parameter | Eucalyptus |

Thermal pretreatment design targets

| | | |
|---|---|---|
| Temperature | ° C. | 190 |
| Residence time | min | 30 |
| Additives | | H₂SO₄ |
| Additive application rate | mg/g ODM | 3 |
| Reactor discharge | wt/wt | 70% |
| moisture | | |

Mechanical pretreatment design targets

| | | |
|---|---|---|
| Compounder speed | rpm | 700 |

Enzymatic hydrolysis targets

| | | |
|---|---|---|
| Slurry concentration | g ODM/g slurry | 20% |
| Hydrolysis time | Hours | 72 |
| Temperature | ° C. | 50 |
| pH | | 5.3 |
| Surfactant type | | N/A |
| Surfactant dosage | mg/g ODM | 0 |
| Enzyme type | | cellulases |
| Enzyme protein dosage | mg/g ODM | 5.8 |
| Hydrolysis yields | | |
| achieved | | |
| Glucan yield achieved⁵ | % of theoretical | 65% |
| Xylan yield achieved⁵ | % of theoretical | 47% |

Fermentation targets

| | | |
|---|---|---|
| Slurry concentration | g ODM/g slurry | 17% |
| Fermentation time | hours | 48 |
| Fermentation temp | ° C. | 32 |
| Fermentation pH | | 5.3 |
| Fermentation nutrients | | |
| Peptone dosage | g/l broth | 4 |
| Yeast extract dosage | g/l broth | 2 |
| Yeast dosage | g/l broth | 2.3 |
| Final ethanol titer | g/l | 44.8 |
| Fermentation yield | % theoretical, | 107% |
| | based on initial | |
| | sugars | |
| Ethanol yield | l/BDMT | 285 |

The measured fermentation yield>100% indicates that significant hydrolysis occurred during fermentation, releasing more sugars for conversion to ethanol.

### Example 6

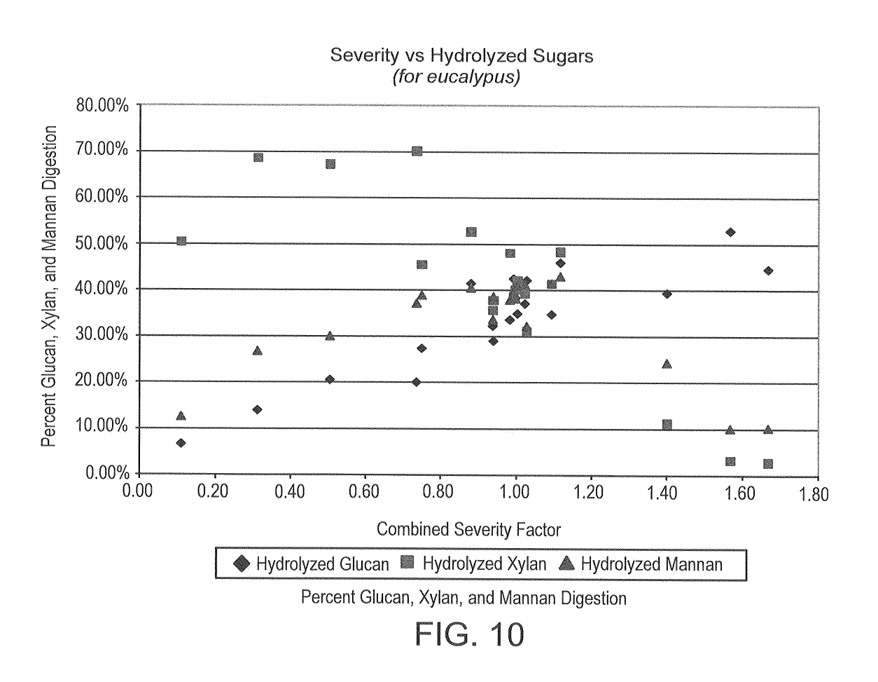

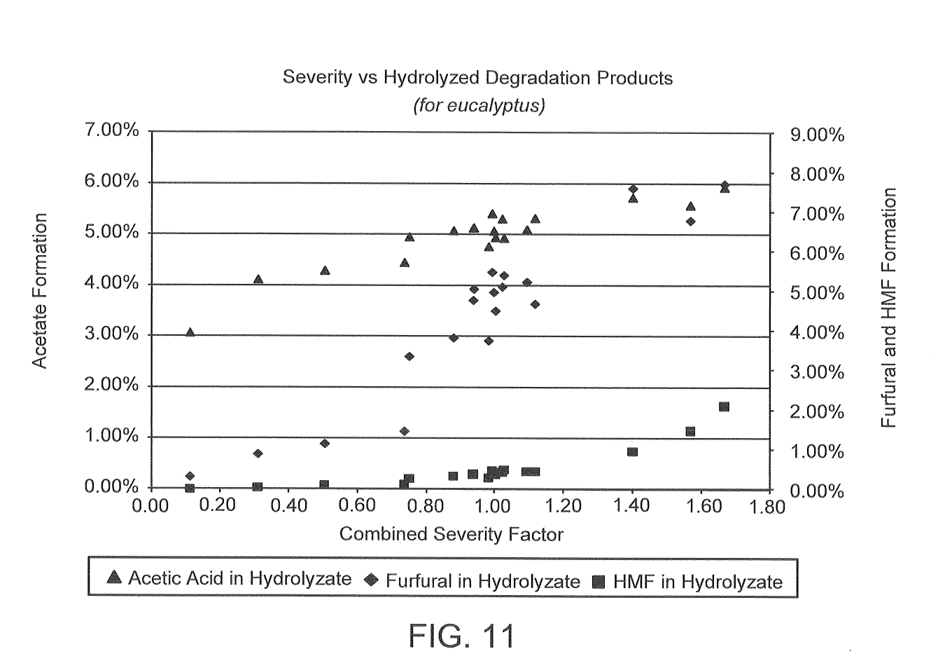

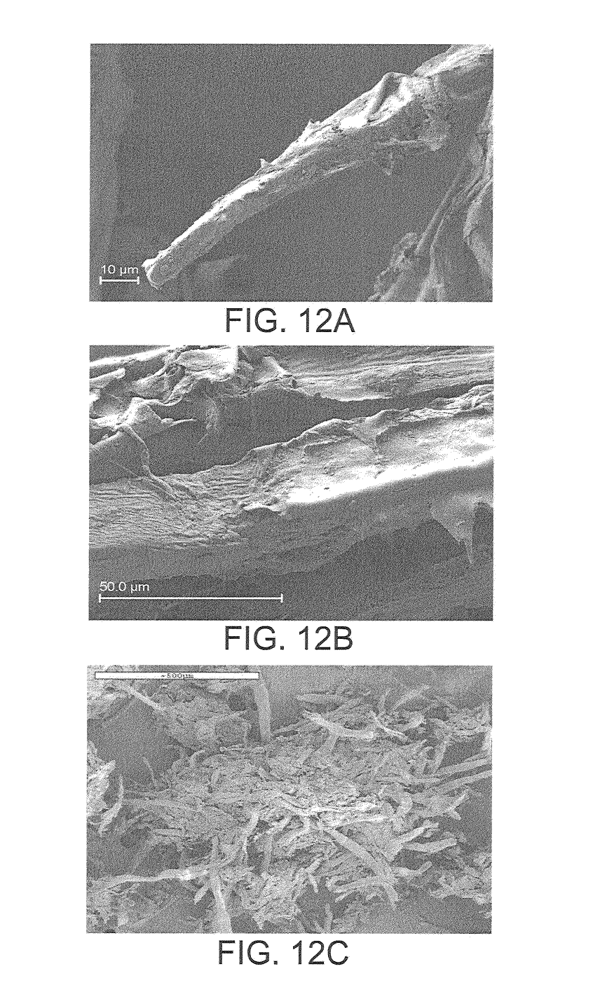

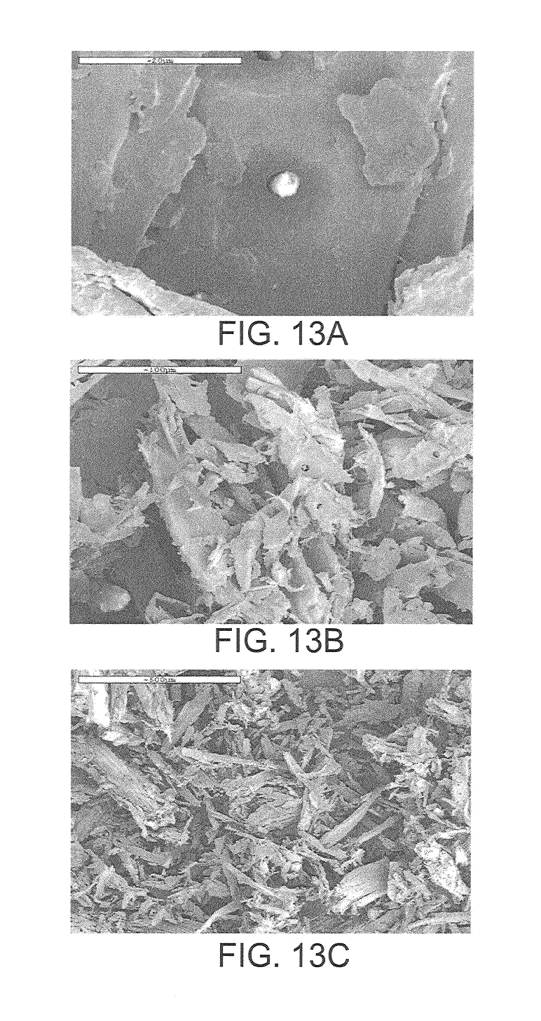

To exemplify the results of the experimental design described in Step 2 of the Biomass Feedstock Testing Protocol, FIGS. 10 and 11 show a typical analytical response of the formation of monomeric sugars and subsequent degradation of those sugars to its degradation products.

### Example 7

Mixed hardwood: Biomass feedstock of mixed hardwood was processed using the foregoing protocol. The material exiting the compounder was collected, hydrolyzed, and analyzed, and the results are shown in Table 6 below:

TABLE 6
Mixed Hardwood

| | |
|---|---|
| Design Parameter | Eucalyptus |

Thermal pretreatment design targets

| | | |
|---|---|---|
| Temperature | ° C. | 183 |
| Residence time | min | 18 |
| Additives | | H₂SO₄ |
| Additive application rate | mg/g ODM | 1 |
| Reactor discharge | wt/wt | 30% |
| moisture | | |

Mechanical pretreatment design targets

| | | |
|---|---|---|
| Compounder speed | rpm | Not available |

Enzymatic hydrolysis targets

| | | |
|---|---|---|
| Slurry concentration | g ODM/g slurry | 20% |
| Hydrolysis time | Hours | 72 |
| Temperature | ° C. | 50 |
| pH | | 5.1 |
| Surfactant type | | Non-ionic |
| Surfactant dosage | mg/g ODM | 20 |
| Enzyme type | | cellulases |
| Enzyme protein dosage | mg/g ODM | 150 |
| Hydrolysis yields | | |
| achieved | | |
| Glucan yield achieved | % of theoretical | 93% |
| Xylan yield achieved | % of theoretical | 52% |

Additional side-by-side testing and sampling for mixed hardwoods was conducted as well for processes involving: (i) hammer milling alone; (ii) hammer milling and mechanical compounding; (iii) hammer milling and thermal reacting; and (iv) hammer milling, thermal reacting, and mechanical compounding. Corresponding glucose and xylose recoveries were determined, as summarized in Table 7 below, and show that the overall best results result from a pretreatment process of the present invention, which involves hammer milling, thermal reacting, and mechanical compounding.

TABLE 7
Mixed Hardwood

| | Glucose | Xylose |
|---|---|---|
| Pretreatment Process | Recovery (%) | Recovery (%) |
| Milling Alone | 4% | 2% |
| Milling + Mechanical Compounding | 28% | 6% |
| Milling + Thermal Reacting | 63% | 49% |
| Milling + Thermal Reacting + | 93% | 52% |
| Mechanical Compounding | | |

### Example 8

Mixed softwood: Biomass feedstock of mixed softwood was processed using the foregoing protocol. The material exiting the compounder was collected, hydrolyzed, and analyzed, and the results are shown in Table 8 below:

TABLE 8
Mixed Softwood

| | |
|---|---|
| Design Parameter | Eucalyptus |

Thermal pretreatment design targets

| | | |
|---|---|---|
| Temperature | ° C. | 190 |
| Residence time | min | 30% |
| Additives | | H₂SO₄ |
| Additive application rate | mg/g ODM | 3 |
| Reactor discharge | wt/wt | 30% |
| moisture | | |

Mechanical pretreatment design targets

| | | |
|---|---|---|
| Compounder speed | rpm | Not available |

Enzymatic hydrolysis targets

| | | |
|---|---|---|
| Slurry concentration | g ODM/g slurry | |
| Hydrolysis time | Hours | 72 |
| Temperature | ° C. | 50 |
| pH | | 5.0 |
| Surfactant type | | Non-ionic |
| Surfactant dosage | mg/g ODM | 20 |
| Enzyme type | | cellulases |
| Enzyme protein dosage | mg/g ODM | 150 |
| Hydrolysis yields | | |
| achieved | | |
| Glucan yield achieved | % of theoretical | 68% |
| Xylan yield achieved | % of theoretical | 35% |
| Mannan yield achieved | % of theoretical | 65% |

Additional side-by-side testing and sampling for mixed hardwoods was conducted as well for processes involving: (i) hammer milling alone; (ii) hammer milling and mechanical compounding; (iii) hammer milling and thermal reacting; and (iv) hammer milling, thermal reacting, and mechanical compounding. Corresponding glucose and xylose recoveries were determined, as summarized in Table 9 below, and show that the overall best results result from a pretreatment process of the present invention, which involves hammer milling, thermal reacting, and mechanical compounding.

TABLE 9
Mixed Softwood

| | Glucose | Xylose | Mannose |
|---|---|---|---|
| Pretreatment Process | Recovery (%) | Recovery (%) | Recovery (%) |
| Milling Alone | 3% | 0% | 0% |
| Milling + Mechanical | 31% | 14% | 0% |
| Compounding | | | |
| Milling + Thermal Reacting | 35% | 30% | 63% |
| Milling + Thermal Reacting + | 68% | 35% | 65% |
| Mechanical Compounding | | | |

All publications, patents, articles, and other references cited and/or discussed in this specification are incorporated herein by reference in their entirety and to the same extent as if each reference was individually incorporated by reference. In case of conflict, the present disclosure controls.

We claim:

1. A process for the thermal-mechanical pretreatment of biomass, comprising the steps of:
(a) subjecting a biomass feedstock including fibers containing cellulose, hemicellulose and lignin, to thermal reaction under conditions exceeding atmospheric pressure, at a temperature exceeding ambient temperature, at a predetermined moisture content and for a predetermined amount of time;
(b) reducing the pressure of said thermal reaction under conditions resulting in explosive decompression of said biomass; and
(c) subjecting said decompressed biomass to axial shear forces to mechanically reduce the size of the fibers of said biomass to obtain treated biomass, wherein said treated biomass has a high level of enzymatic digestability and a low concentration of degradation products.

2. The process of claim 1, wherein said thermal reaction includes the introduction of live steam.

3. The process of claim 1, wherein the pH of said thermal reaction is adjusted to acidic conditions.

4. The process of claim 3, wherein said acidic conditions include a pH of from 1.0 to 6.0.

5. The process of claim 4, wherein said acidic conditions include a pH of from 2.5 to 4.0.

6. The process of claim 3, wherein said acidic conditions are obtained by introducing an acid selected from the group consisting of sulfuric, nitric, phosphoric, hydrochloric, acetic or lactic acids, or mixtures thereof.

7. The process of claim 1, wherein said thermal reaction exceeds 1.0 minutes.

8. The process of claim 7, wherein the thermal reaction takes place from about 10 minutes to about 90 minutes.

9. The process of claim 1, wherein the thermal reaction takes place under pressure of from about 70 psia to about 225 psia.

10. The process of claim 1, wherein the thermal reaction takes place at a temperature of from about 150° C. to about 200° C.

11. The process of claim 1, wherein steam released from said explosive decompression is recovered in a flash tank.

12. The process of claim 1, wherein said explosive decompression reduces the pressure of said thermal reaction to about 5 to about 32 psia.

13. The process of claim 1, wherein said explosive decompression reduces the temperature of said biomass to about 70 to 125° C.

14. The process of claim 1, wherein said axial shear forces are provided by introducing said biomass to a compounder.

15. The process of claim 14, wherein said compounder includes a conveyor zone and a shear zone.

16. The process of claim 14, wherein said compounder includes multiple shear zones separated by conveyor zones.

17. The process of claim 15, wherein said shear zone includes kneading and high surface area elements.

18. The process of claim 14, wherein said compounder includes a single screw.

19. The process of claim 14, wherein said compounder is a twin screw, counter-rotating compounder.

20. The process of claim 14, wherein said compounder is a twin screw, co-rotating compounder.

21. The process of claim 15, wherein said compounder includes downstream from said shear zone, a devolatilization zone for venting steam and heat.

22. The process of claim 21, wherein said compounder includes downstream from said devolatilization zone, a quench zone for introducing fluids to the biomass within the compounder.

23. The process of claim 22, wherein said compounder includes downstream from said quench zone, an enzyme mixing zone for introducing enzymes to said biomass within said compounder.

24. The process of claim 23, wherein said compounder includes downstream from said enzyme mixing zone a slurry mixing zone for adjusting the consistency of said biomass by the introduction of water within said compounder.

25. The process of claim 1, further comprising an enzyme treatment step to convert said treated biomass to monomeric sugars.

26. The process of claim 1, further comprising a fermentation step to convert said monomeric sugars to ethanol.

27. The process of claim 11, wherein said steam captured in said flash tank includes degradation products separated from said biomass.

28. The process of claim 27, wherein said degradation products are selected from the group consisting of furfural and acetic acid and mixtures thereof.

29. The process of claim 1 wherein the concentration of said degradation compounds in said treated biomass is less than 1% of dry biomass feedstock, by weight.

30. The process of claim 1, wherein said predetermined moisture level is from 40% to 80%.

31. The process of claim 30, wherein said moisture level is from 50 to 75%.

32. The process of claim 31, wherein said moisture level is from 60 to 75%.
